(19) United States
(12) Patent Application Publication
(10) Pub. No.: US 2026/0364406 A1
(43) Pub. Date: Nov. 19, 2026

(54) Item of Sporting Equipment With Integrated Muscle Stimulation System and Activation Method

(71) Applicant: Bayerische Motoren Werke Aktiengesellschaft, Muenchen (DE)

(72) Inventors: Matthias FRANZ, Neubeuern (DE); Marius MATZIG, Muenchen (DE)

(21) Appl. No.: 19/515,530

(22) PCT Filed: Nov. 6, 2024
(86) PCT No.: PCT/DE2024/100938

§ 371 Date: Mar. 2, 2026

(30) Foreign Application Priority Data
Nov. 14, 2023 (DE) 10 2023 131 641.9

(51) Int. Cl. A63B 24/00; A63B 22/00; A63B 22/06

(52) U.S. Cl. CPC A63B 24/0087; A63B 22/0076; A63B 22/0605; A63B 2213/004; A63B 2213/007; A63B 2225/50

(57) ABSTRACT

An item of sporting equipment with an integrated muscle stimulation system, comprising one or more actuators on at least one region at which a contact surface with a user is provided, the actuators being designed to carry out muscle stimulation. At least one control device is designed to activate the actuators, and at least one energy supply unit is designed to supply electrical energy to the actuators and the control device. The control device is designed to activate the actuators and stimulate the user's muscles in such a way that a movement of the user for operating the item of sporting equipment is supported.

### BACKGROUND AND SUMMARY

This disclosure relates to an item of sporting equipment with a muscle assistance system and an activation method.

Systems which assist a user, for example, in training by emitting EMS pulses to their muscles are already known. For this purpose, a plurality of pieces of clothing having corresponding equipment is already known, as disclosed, for example, in DE 10 2014 108 316 A1, which enables assistance when riding bicycles. Corresponding pieces of clothing which assist the user are also disclosed in US 2023/0226345 A1 or WO 2016/131936A2. EMS devices which actively train and strengthen the musculature are also already known for training in the fitness studio.

In all applications, the user has to wear corresponding pieces of clothing or electrodes have to be stuck to the skin. This can be cumbersome. It is therefore an object of this disclosure to provide an improved item of sporting equipment with a muscle assistance system.

An item of sporting equipment with integrated muscle stimulation system is proposed, wherein the item of sporting equipment comprises: one or more actuators on at least one area at which a contact surface to a user is provided, wherein the actuators are configured to carry out a muscle stimulation, at least one control device which is configured to activate the actuators, at least one energy supply unit which is configured to supply the actuators and the control device with electrical energy, wherein the control device is configured to activate the actuators such that they stimulate muscles of the user such that a movement of the user to carry out an operation of the item of sporting equipment is assisted.

Moreover, it is provided that the actuators are configured to carry out an electromagnetic stimulation or a functional magnetic stimulation.

Moreover, it is provided that the item of sporting equipment is a bicycle or a rowing machine or a fitness device.

Furthermore, an activation method for a muscle stimulation system arranged in the item of sporting equipment is proposed, wherein the stimulation of the muscles of the user takes place as soon as an operation of the item of sporting equipment or a start signal given by the user has been detected.

Moreover, it is provided that correctness of the operation provided for the item of sporting equipment is monitored and it is corrected if needed by adjusting the stimulation.

Moreover, it is provided that the user can perform an adjustment of the intensity of the stimulation.

Furthermore, a computer program product is proposed, having program code for carrying out the method when it is executed on at least one control device of the item of sporting equipment.

Further features and advantages of the disclosure result from the following description of exemplary embodiments, on the basis of the figures, which show details according to the disclosure, and from the claims. The individual features can each be implemented individually as such or in groups in arbitrary combination in a variant of the disclosure.

### BRIEF DESCRIPTION OF THE DRAWING

FIG. 1 shows a schematic representation of a bicycle having a stimulation system for muscle assistance according to an embodiment of the present disclosure.

### DETAILED DESCRIPTION OF THE DRAWING

As already mentioned, pieces of clothing are already known which assist a user 2 in their training, among other things when riding bicycles. However, assistance can also take place with other types of sport or devices.

The user is presently also assisted when riding a bicycle by an electric drive of the bicycle. The force applied by the user to the pedal is boosted here by the electric drive as the assistance. Alternatively, the drive can also be set in the opposite direction to the pedaling direction, so that a training effect results. However, no stimulation takes place here if the user does not wear a corresponding piece of clothing.

According to the disclosure, it is proposed that an item of sporting equipment with integrated (muscle) stimulation system for muscle assistance be provided, wherein the actuators are arranged on a contact surface to a user depending on the item of sporting equipment.

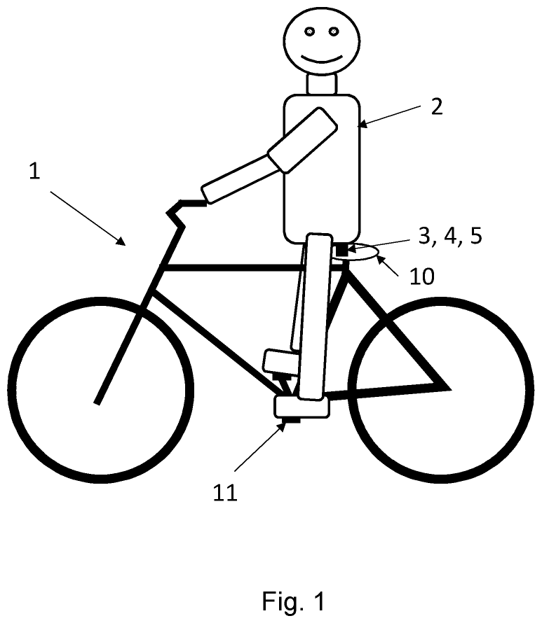

Both a bicycle 1, as shown by way of example in FIG. 1, and also a fitness device or a rowing machine can be used as the item of sporting equipment. It is important that the muscle group to be assisted is applied to a part of the item of sporting equipment so that a stimulation of the muscle group can take place.

On the basis of the example of the bicycle 1 (with or without electric drive) shown in FIG. 1, it can be provided that the actuators 3 used for the stimulation are arranged in the area of the bicycle saddle 10, thus the buttocks musculature and possibly even the thigh musculature, in particular the rear thigh musculature (leg extensors). A control device 4 and an energy supply 5 are likewise provided here. As soon as the control device 4 either detects pedaling of the pedals 11 of the bicycle 1 or a user 2 gives a starting signal (for example, pressing a button), it activates the actuators 3 in the bicycle saddle 10, in order to apply a stimulation of the buttocks musculature and possibly a part of the leg musculature thereto. The user 2 can apply more force when pedaling due to the stimulation of the musculature, so that they move forward faster. The musculature is also strengthened at the same time. Depending on the degree of fitness, it can even be possible that the user 2 no longer requires an electric drive, and can thus replace the bicycle 1, which is very heavy due to the drive, with a lighter bicycle 1, which has the stimulation system. In some embodiments, the control device 4 may comprise a processor configured to execute software, instructions, and/or logic stored on a memory.

In the case of a rowing machine, the stimulation system can likewise be provided on a seat surface of the machine, but also additionally or alternatively in a backrest or an armrest, etc. In the case of other fitness devices, the stimulation system can likewise be provided on a seat surface, a backrest, an armrest, etc.

The energy supply required for the actuators 3 of the stimulation system and the control device 4 can take place either via an energy supply of the item of sporting equipment or via an accumulator 5 or both. In the case of an accumulator 5, it can be charged by operating the item of sporting equipment (by recuperation), wherein in this case the stimulation cannot take place simultaneously, but rather only when the accumulator 5 is sufficiently charged. However, it can also be directly connectable to an external power supply for charging, or the accumulator is removable and can be brought to a charging station and/or replaced by another accumulator.

It is clear from the description that the stimulation system integrated in the item of sporting equipment contributes to strengthening the musculature of the user 2 and using the item of sporting equipment more effectively. It is not necessary in this case for the user 2 to wear special clothing having integrated electrodes or for electrodes to have to be arranged on the skin. Rather, the stimulation effect has been proven to also extend through the normal clothing of the user. Due to the control device 4 provided, it is also not necessary to select a program for the stimulation system, since it is known on the basis of the type of the item of sporting equipment which muscle groups are to be stimulated, due to which a stimulation can be preset.

In one embodiment, the user 2 can set the intensity of the stimulation, preferably for each muscle group that can be stimulated.

In one embodiment, it is provided that the operation of the item of sporting equipment, thus the exercise performed, is monitored, for example, by the control device 4. The performance of the movement of the user is compared here to a stored desired movement. In the case that the user 2 deviates therefrom (the permitted degree of deviation can be specified), the stimulation device intervenes correctively in that, for example, a muscle which is activated too little by the user 2 is stimulated more strongly. The correct use of the item of sporting equipment and the correct performance of the exercise can thus be promoted. A weaker muscle part of the user 2 can thus also be strengthened.

The movement performance of the user can thus be improved by the stimulation system integrated in the item of sporting equipment and muscle training can be carried out without the user having to wear special clothing, or electrodes having to be applied to their skin.

The stimulation can take place by way of EMS (electromagnetic stimulation) or FMS (functional magnetic stimulation).

### LIST OF REFERENCE SIGNS

1 vehicle
10 vehicle seat
11 pedal
2 user
3 actuator
4 control device
5 accumulator

1-7. (canceled)

8. An item of sporting equipment with integrated muscle stimulation system, the item of sporting equipment comprising:
one or more actuators on at least one area at which a contact surface to a user is provided, wherein the actuators are configured to perform a muscle stimulation,
at least one control device configured to activate the actuators, and
at least one energy supply unit configured to supply the actuators and the control device with electrical energy, wherein the control device is configured to activate the actuators to stimulate the muscles of the user such that a movement of the user to perform an operation of the item of sporting equipment is assisted.

9. The item of sporting equipment according to claim 8, wherein the actuators are configured to carry out an electromagnetic stimulation or a functional magnetic stimulation.

10. The item of sporting equipment according to claim 8, wherein the item of sporting equipment is a bicycle or a rowing machine or a fitness device.

11. An activation method for a muscle stimulation system arranged in an item of sporting equipment according to claim 8, wherein the stimulation of the muscles of the user takes place as soon as an operation of the item of sporting equipment or a starting signal given by the user has been detected.

12. The method according to claim 11, wherein a correctness of the operation provided for the item of sporting equipment is monitored and is corrected if needed by adjusting the stimulation.

13. The method according to claim 11, wherein the user can perform an adjustment of the intensity of the stimulation.

14. A non-transitory computer readable medium having instructions that, when executed by a processor, perform the method of claim 11.
